(19) United States
(12) Patent Application Publication
(10) Pub. No.: US 2011/0165203 A1
(43) Pub. Date: Jul. 7, 2011

(54) A TOPICAL COMPOSITION COMPRISING THE ISOLATED NON-POLAR LIPID FRACTION OF LANOLIN4

(76) Inventors: Johanna Aaltje Bouwstra, Leiden (NL); Maria Helen Ponec, Leiden (NL); Marion Henrica Monique Oudshoorn, Utrecht (NL); Robert Rissmann, Leiden (NL); Wilhelmus Everardus Hennink, Utrecht (NL)

(21) Appl. No.: 12/994,088

(22) PCT Filed: May 27, 2009
(86) PCT No.: PCT/EP2009/003848

§ 371 Date: Feb. 18, 2011

(30) Foreign Application Priority Data
Jun. 2, 2008 (GB) 0809983.0

(51) Int. Cl. A61K 31/20; A61K 31/56; A61K 9/00; A61P 31/00; A61P 17/02; A61P 17/16

(52) U.S. Cl. 424/400; 514/558; 514/171

(57) ABSTRACT

A topical composition comprising the isolated non-polar lipid fraction of lanolin, and one or more additional compounds selected from ceramides, cholesterol and free fatty acids.

This invention relates to synthetic compositions which mimic certain properties of natural Vernix Caseosa (herein Vernix).

Vernix is a lipid-rich naturally occurring skin protectant composed of sebum, epidermal lipids and desquamated dead epithelial cells. It covers the skin of the developing fetus in utero while the fetus is completely surrounded by amniotic fluid. Natural Vernix comprises about a 10% lipid fraction by weight, about a 10% protein fraction by weight, and about an 80% volatile fraction by weight. The lipid matrix undergoes a transition to a more fluid form at physiological temperatures. Vernix provides a covering for the skin during gestation. The structure of Vernix resembles that of the stratum corneum except that it lacks multiple desmosomal connections, and the lipid matrix forms a less ordered lipid organisation. Consequently, Vernix exhibits a viscous fluid character.

The lipid component of Vernix has been reported in J. Invest. Dermatol. 78:291 (1982): Lipids 6:901 (1972); J. Clin. & Lab. Investigation 13:70 (1961); J. Invest. Dermatol., 44:333 (1965); U.S. Pat. No. 5,631,012; and J. Invest. Dermatol. 26:1823 (2006); each of which is incorporated by reference herein in its entirety. Lipids, defined herein as fats or fat-like substances, include lecithin and other phospholipids, squalene, waxes, wax esters, sterol esters, dihydroxy wax esters, triglycerides, free fatty acids, free sterols and ceramides. The latter consists of a non-hydroxy, α-hydroxy or ω-hydroxy fatty acid linked to a base of either sphingosine, phytosphingosine, or 6-hydroxy sphingosine. In addition a C18 fatty acid (a high percentage of which is linoleic acid) is linked to the ω-hydroxy fatty acid moiety of the ceramides.

In Vernix, all combinations of the fatty acid linked to the base have been identified. The fatty acid chain length of the ceramides varies between C12 and C34. The free fatty acid chain length varies between C12 and C28. The free fatty acids consist of four classes, namely straight chain saturated, straight chain unsaturated, branched chain saturated, and branched chain unsaturated. The lipid fraction may contain, with the relative approximate percentages indicated, squalene (6%), ceramides (5%) aliphatic waxes and sterol esters (42%), dihydroxy wax esters (6%), triglycerides (36%), cholesterols (4%), and free fatty acids (2%). The fatty acids within the aliphatic waxes may be branched and the branched fatty acids may be methylated.

Because of its anticipated skin maturation and protectant properties, Vernix appears to have promise as a clinically effective therapeutic agent. Application of Vernix to clinical use, however, has been limited by the difficulty in obtaining samples of sufficient volume, and the possibility of disease transmission.

Regarding its physical properties, Vernix in utero is a tractable semi-solid, whereas Vernix ex utero is a relatively intractable compound with a very thick consistency. Vernix is not completely soluble in conventional solvents such as absolute ethanol, 95% ethanol, 2-propanol, and combinations of chloroform and methanol. Thus, controlled and uniform administration of Vernix to a surface is difficult. It has been reported that the surfactant polysorbate 80 (Tween 80) may solubilise Vernix, but Tween 80 is toxic to living cells and therefore cannot be used clinically. Isolated reports disclose Vernix directly scraped from a newborn for smearing over wounds (U.S. Pat. No. 1,718,947A) or an artificial lipid composition as a cosmetic moisturizer (U.S. Pat. No. 5,631,012).

US-A-2005/0232890 describes synthetic Vernix compositions. According to this teaching, the compositions may comprise a plurality of hydrated synthetic cells dispersed in a liquid matrix in effective amounts to provide a minimum surface free energy of about 20 dynes/cm to the skin surface. In more specifically described embodiments, the compositions are said preferably to comprise about 5% to about 30 wt % protein and about 5% to 30 wt % lipid component, the lipid component optionally comprising compounds such as cholesterol esters, ceramides, triglycerides, cholesterol, free fatty acids, phospholipids, wax esters, squalene, dihydroxy wax esters, and cholesterol sulphate. The remaining composition may be a simulated cell/water component; suitable simulated cells are said to comprise cubosomes, phospholipid lipsomes, nanoparticles, microparticles, colloidosomes, non-phospholid liposomes (Catezomes®), or cultured cells. Suitable uses of the synthetic compositions are said to include protecting the surface of either normal (e.g. intact) or compromised skin (e.g. wounded, abraded or cut) from the effects of water exposure, or to bring about skin growth, maturation, or healing when applied to a wound or ulcer, or to provide a barrier, such as a water repellent or moisturizing function, when applied to normal, chapped or irritated skin.

WO2006/078245 describes synthetic vernix compositions which are said optionally to contain (whole) lanolin as one of a variety of possible lipid components. The teaching apparently contemplates levels of lanolin no higher than 3 wt %.

We have devised a different form of synthetic Vernix composition which mimics some of the benefits of natural Vernix, and may particularly be used to promote skin barrier recovery.

In a first aspect of the invention, there is provided a topical composition comprising the isolated non-polar lipid fraction of lanolin, and one or more additional compounds selected from ceramides, cholesterol and free fatty acids.

According to a further aspect of the invention, there is provided a topical composition comprising the isolated non-polar lipid fraction of lanolin, and one or more additional compounds selected from ceramides, cholesterol and free fatty acids for use as an active therapeutic substance.

According to yet a further aspect, there is provided a topical composition comprising the isolated non-polar lipid fraction of lanolin, and one or more additional compounds selected from ceramides, cholesterol and free fatty acids for use in improving barrier formation and repair, to act during delivery as a lubricant, to exhibit anti-infective, anti-oxidant or skin hydrating benefits, or protect skin post-natally, or to enhance wound healing.

The additional compounds selected from ceramides, cholesterol and free fatty acids added to compositions according to the invention are in addition to any which remain in the non-polar lipid fraction of lanolin, though in practice it has been found that negligible amounts if any of these compounds remain in the non-polar lanolin fraction.

The non-polar fraction of lanolin typically comprises those lipids which are less polar than triglycerides.

Suitable free fatty acids for use according the invention typically comprise straight chain saturated, straight chain mono- or di-unsaturated or branched chain fatty acids with a chain length of 14 to 26 carbon atoms. Conveniently the fatty acids are C16 or C18; conveniently they are saturated or mono-unsaturated. Topical compositions according to the invention typically comprise 0.1 wt % to 10 wt %, preferably 0.5 wt % to 5 wt %, most preferably 1.5 wt % to 3.0 wt % of the composition of free fatty acids.

Conveniently, the lanolin is anhydrous lanolin.

Conveniently, the additional compounds present in the composition or used according to the invention can comprise any pair of compounds selected from ceramides, cholesterol and free fatty acids, i.e. ceramides and cholesterol, ceramides and fatty acids, and cholesterol and fatty acids. In a preferred embodiment the additional compounds present in the composition or use according to the invention comprise all three compounds (i.e. ceramides, cholesterol and free fatty acids).

Conveniently, the topical compositions according to the invention may comprise more than 30% by weight of the composition of lipids (i.e. the non-polar lipid fraction isolated from lanolin, plus the additional compounds).

Cholesterol is typically present in topical compositions at a level of 0.1 wt % to 10 wt %, preferably 0.5 wt % to 3 wt %.

According to a further aspect of the invention, there is provided a topical composition comprising a plurality of hydrated synthetic cells dispersed in lipids, conveniently a lipid matrix, wherein the synthetic cells comprise a hydrogel. Conveniently, the lipids comprise the isolated non-polar lipid fractional of lanolin, and one or more additional compounds selected from ceramides, cholesterol and free fatty acids.

The non-polar lipid component of the composition which is isolated from lanolin may conveniently be isolated by chromatographic techniques, such as column chromatography. It has been found that the non-polar lipid component may be further broken down into a fraction which is rich in sterol esters and wax esters, a fraction which is rich in dihydroxy wax esters, and a fraction which is rich in sterol esters, wax esters and dihydroxy wax esters. The separation of these fractions can readily be carried out by those skilled in the art using column chromatography. In certain embodiments, the non-polar lipid component used in compositions according to the invention may be that isolated fraction which is rich in sterol esters, wax esters and dihydroxy wax esters, optionally absent the other two non-polar fractions named above. Conveniently, topical compositions according to the invention comprise more than 3 wt % non-polar fraction of lanolin, conveniently at least 5 wt %, and in some embodiments at least 8 wt %, conveniently at least 10 wt % or 15 wt %. Conveniently the topical composition comprises no more than 60 wt %, and in some embodiments no more than about 50 wt %, no more than about 40 wt % or no more than about 30 wt % non-polar fraction of lanolin. Suitable isolated purified non-polar lipid fractions may also be commercially available, for example from Croda under the trade name Super Sterol Esters. The preferred non-polar lipid fractions and the resulting lipid mixtures utilised have similar thermotropic properties compared to natural Vernix.

In compositions according to the invention, the composition may additionally comprise water. In some aspects, the composition may comprise more than 10% by weight, conveniently more than 15% by weight, conveniently more than 20% by weight, conveniently more than 30% by weight lipid. In other aspects, the composition according to the invention may comprise more than 35%, more than 40% by weight, more than 45% by weight or more than 50% by weight of the lipid. Compositions according to some aspects of the invention may comprise up to 100% by weight lipid.

Compositions according to the invention may comprise ceramides, which may be natural or synthetic in origin: Molecular structures of synthetic ceramides, which resemble pig ceramide and closely mimic human ceramide, are reported in “Lipid mixtures prepared with well-defined synthetic ceramides closely mimic the unique stratum corneum lipid phase behaviour,” de Jager, Goods, Ponec and Bouwstra, J. Lipid Res 46 (2005), 2649-2656, the contents of which are incorporated by reference herein in their entirety. Ceramide subclasses are denoted by a letter-based system introduced by Motta et al, “Ceramide composition of psoriatic scale”, Biochimica Biophysica Acta 1182 (1993) 147-151, the contents of which are incorporated herein by reference, with the number of carbon atoms in the acyl chain being C16, C24 or C30. Ceramides are typically present in the topical composition at levels of 0.01 wt % to 5 wt %, more conveniently 0.1 wt % to 3 wt %.

Synthetic ceramides tend to be characterised by a relatively uniform chain length. A suitable synthetic ceramide blend for use according to the invention comprises EOS(C30) linoleate, NS(C24), NP(C24), AS(C24), AS(C16) and AP(C24) at weight ratios of 14.6, 20.8, 8.3, 8.3, 16.7 and 31.3% respectively.

Topical compositions according to the invention may additionally comprise added squalene, typically if present at a level of 0.1% to 10 wt %, preferably 0.5 to 5 wt %, conveniently 1 to 3 wt % by weight of the topical composition and/or added triglycerides, typically if present at a level of at least 1 wt %, preferably at least 2 wt %, preferably at least 3 wt %, preferably at least 5 wt %, and in some embodiments preferably at least 7 wt %, preferably at least 10 wt %, and maybe at least 15 wt % of the topical composition Conveniently, the triglyceride may comprise no more than 40 wt %, conveniently no more than 25 wt %, conveniently no more than 20 wt % by weight of the topical composition. Suitable triglycerides may comprise straight chain saturated or mono-unsaturated or branched chains with a chain length of C14-C26.

In a preferred aspect, to try to optimise the lipid phase behaviour, especially in compositions comprising synthetic (e.g. relatively uniform chain length) ceramides, the triglycerides may comprise at least 65% by weight, preferably at least 70%, preferably at least 75%, 80%, 85% or 90% by weight of the total triglyceride content of triglycerides which comprise unsaturated fatty acids. In such circumstances, a particularly preferred triglyceride blend comprises equal weight ratios of triglycerides which have been esterified with 16:1, 18:1 and 24:1 fatty acids.

Compositions according to the invention may optionally contain synthetic cells. Suitable synthetic cells may include those described in US-A-2005/0232890, and to this end the content of this application in as far as it relates to the nature, preparation and use of synthetic cells is incorporated herein by reference in its entirety.

Conveniently, the synthetic cells are homogenously distributed in the topical composition.

In some aspects, a preferred synthetic cell which may be used in compositions according to the invention may comprise a hydrogel. As used herein, the term “hydrogel” refers to gels whose liquid constituent comprises water. According to some aspects, the liquid constituent of the hydrogel may comprise greater than 85% by weight, greater than 90% by weight, greater than 95% by weight, greater than 99% by weight, or even close to 100% by weight water. Other fluids which may be incorporated in the hydrogel include solvents which are miscible with water, including polyols such as glycerol and propylene glycol, short chain (e.g. C₁ to C₄) alcohols such as ethanol and isopropanol, DMSO, and so on.

Hydrogels used in compositions according to the invention may comprise 1 to 95% by weight of the hydrogel water, preferably 10 to 80% by weight of the hydrogel water, and in some embodiments 20 to 60% by weight, preferably 30% to 60% by weight of the hydrogel of water. If utilised, hydrogels may be present in topical compositions according to the invention at a level of up to 90 wt % of the topical composition, conveniently 1 wt % to 85 wt % by weight of the topical composition.

Hydrogels are known in the art, as is their preparation. References which discuss hydrogels and their preparation include:
Hennink W E, van Nostrum C F. Novel cross-linking methods to design hydrogels. Adv Drug Deliv Rev 2002; 54(1): 13-16;
Hoffman A S. Hydrogels for biomedical applications. Adv Drug Deliv Rev 2002; 54(1): 3-12;
Peppas N A, Bures P, Leobandung W, Ichikawa H. Hydrogels in pharmaceutical formulations. Eur J Pharm Biopharm 2000; 50(1): 27-46;
Hennink W E, van Nostrum C F, Crommelin D J. Bezemer J M. Hydrogels for the controlled release of proteins, in: Kwon G. S (Ed), Polymeric drug delivery systems, Taylor & Francis Group, Boca Raton, Fla. 2005; 148: 215-249; and
Jeong S H, Fu Y, Park K. Hydrogels for oral administration, in: Kwon G. S. (Ed.), Polymeric drug delivery systems, Taylor & Francis Group, Boca Raton, Fla. 2005: 148: 195-214;

the contents of which are all incorporated herein by reference in their entirety.

Hydrogels are hydrophilic, cross-linked polymeric materials, which are capable of absorbing large amounts of water while preserving a three-dimensional (3-D) network structure. To prevent the polymer chains from dissolving in an aqueous environment, cross-links have to be present in the hydrogels. This can be achieved by either physical or chemical cross-linking of the hydrophilic polymers. The method of cross-linking used is dependent on the specific application of the hydrogels; physical cross-linked gels can be formed under mild conditions and can be reversibly broken, although their mechanical strength is often weak, whereas chemical cross-linking results in the formation of reproducible, well-defined gels with a high mechanical strength, though chemical cross-linking agents might damage loaded substances. In addition to the cross-linking method, the degradability of the polymeric networks as well as the formed degradation products can be tailored.

Degradability can be established by introducing labile bonds whereas the formed degradation products can be tailored by selecting the appropriate hydrogel building blocks. As a result a wide and diverse range of polymers, depending on the required properties, can be used to prepare hydrogels.

The composition of the polymers used according to the invention can be divided into two groups: natural polymers, such as hyaluronic acid, dextran and chitosan, and synthetic polymers, including poly(ethylene glycol) (PEG) and poly(hydroxyethyl methacrylate) (pHEMA).

A preferred type of polymer which may be used for preparing suitable hydrogels for use according to the invention are derivatised cross-linkable acrylate or methacrylate polymers, for example photocross-linkable methacrylated hyperbranched polyglycerol materials. However any polymer which is capable of forming a hydrogel may be suitable, including natural and synthetic biocompatible polymers, which polymers can be cross-linked either via chemical or physical bonds.

Hyperbranched polyglycerol materials are known in the art; references relating to their synthesis, biocompatibility and network formation include:
Haag R. Sunder A. Stumbe J. “An approach to glycerol dendrimers and pseudo-dendritic polyglycerols.” J. Am Chem Soc 2000; 122: 2954-2955;
Kainthan R K, Muliawan E B, Hatzikiriakos S G, Brooks D E. “Synthesis, characterization, and viscoelastic properties of high molecular weight hyperbranched polglycerols.” Macromolecules 2006; 39: 7708-7717;
Kainthan R K, Hester S R, Levin E, Devine D V, Brooks D E. “In vitro biological evaluation of high molecular weight hyperbranched polyglycerols.” Biomaterials 2007; 28(31): 4581-4590;
Kainthan R K, Brooke D E. “In vivo biological evaluation of high molecular weight hyperbranched polyglycerols.” Biomaterials 2007; 28(32): 4779-4787;
Oudshoorn M H M, Rissmann R, Bouwstra J A, Hennink W E. “Synthesis and characterization of hyperbranched polyglycerol hydrogels.” Biomaterials 2006; 27(32): 5471-5479; and
Oudshoorn M H M, Penterman R, Rissmann R, Bouwstra J A, Broer D J, Hennink W E. “Preparation and characterization of structured hydrogel microparticles based on crosslinked hyperbranched polyglyerol.” Langmuir 2007; 23(23): 11819-11825;

the contents of which are incorporated herein by reference in their entirety.

Hyperbranched polyglycerols used according to the invention may be prepared using anionic ring-opening multi-branching polymerisation of glycidol. Hyperbranched polyglycerols with molecular weights ranging from 1,000 to 30,000 g/mol and with narrow polydispersities (M_{w}/Mₙ<1.5) were readily synthesised. Hyperbranched polyglycerols consist of an inert polyether backbone with functional hydroxyl groups at every branch end. This structural feature resembles the well known poly(ethylene glycol) that is used in a variety of biomedical applications. The polyether backbone of hyperbranched polyglycerol, taking the biocompatibility of aliphatic polyether structures such as poly(ethylene glycol) into account, makes hyperbranched polyglycerol a suitable polymer for use as described herein.

Homogenisation of the synthetic cells within the topical composition can at least on a relatively small scale be obtained by mixing the composition with a Topitec (WEPA, Germany) automatic ointment mixer at a speed of 400 rpm for 5 minutes. This resulted in a homogenous dispersion of synthetic cells which were not damaged.

Compositions according to the invention have been found to mimic ideally at least the thermotropic behaviour of natural Vernix. In addition, the compositions according to the invention may provide a number of benefits. They may be suitable for promoting barrier formation and repair when applied to disrupted (e.g. wounded or abraded) skin, and as such are suitable for formulation into a topical composition adopted for such application. They may also be suitable to formulate into a barrier cream. They may also be suitable for application to newborn babies, for example to act as anti-infective, anti-oxidant and skin hydrating benefits, as well as protecting and cleansing skin post-natally. The benefits may be obtained particularly by low birth weight infants, for example premature babies, which may have a deficient barrier function or a deficiency of Vernix, but may also be beneficial to adults and children to enhance wound healing. Compositions according to the invention may also be suitable for use as a protective layer on superficial wounds where for example the stratum corneum is largely or completely absent, and to restore the hydration level of skin in which the stratum corneum is still largely present. They may also be used to hydrate and repair the skin barrier in diseased skin.

In an embodiment, the synthetic cells used in compositions according to the invention can be pre-coated with one or more lipids.

Where pre-coated synthetic cells are utilised, the method used was that described by De Geest, Stubbe, Jonas, Van Thienen, Hinrichs, Demeester, De Smedt, “Self-exploding lipid-coated microgels”, Biomacromolecules 2006; 7(1): 373-379, the content of which is incorporated herein by reference. Suitable coatings for synthetic cells include liposomal coatings, for example liposomes made with dioleoyl phosphatidylcholine lipids, and dioleoyl trimethylammoniumpropane lipids. In preparation, the liposomes were mixed with the synthetic cells and coated onto the surface of the cells; the liposomes then fused to form a multilayer coating. Suitable coatings also include ceramide coatings and chemically bound lipid coatings, which may be attached by either covalent chemical bonding (involving e.g. chemical cross-linking) or physical interactions. In an alternative contemplated aspect, the lipid coating can be adhered to the synthetic cell using an intermediate layer (i.e. between the synthetic cell and the lipid coating) of an amphiphilic polymer which react or interact on one end (e.g. the hydrophilic end) with the synthetic cell, and on the other (e.g. hydrophobic) end with the lipid coating; again such interactions can be chemical or physical in nature

If utilised, synthetic cells (e.g. hydrogels) used in compositions according to the invention may incorporate a benefit material. Such benefit materials may include biologically active compounds, pharmaceutical products and compounds, proteins, natural moisturizing factors, DNA, (si)RNA, hygroscopic compounds, and so on. The synthetic cell (e.g. hydrogel) can also be degradable, to release the benefit material so incorporated.

Synthetic cells used in compositions of the invention will typically have dimensions such as lengths, widths or depths in the range 0.1 microns to 100 microns, preferably 1 micron to 80 microns, conveniently 10 to 50 microns, conveniently 20 to 30 microns.

Compositions according to the invention may also comprise proteins, though in some embodiments suitable compositions may be produced which are protein-free. If present in the composition, protein may typically comprise 0.1% to 35% by weight, typically 0.1% to 20% by weight of the lipid matrix.

Suitable sources of proteins include commercially available proteins, recombinant protein, synthetic proteins, or epidermally derived proteins, keratin, filaggrin, epidermal growth factor, surfactant associated protein -A, -B or -D, peptide natural moisturizing factor.

In certain embodiments, compositions according to the invention may have a weight ratio of the hydrated synthetic cell (e.g. polymer plus water) to lipid of 10:1 to 1:1, conveniently 8:1 to 1:1, conveniently 5:1 to 2:1.

Compositions according to the invention may typically be prepared by dispersing hydrated polymer particles in a minimal volume of “free” water prior to the addition of the lipid component, and mixing

Compositions according to the invention may optionally comprise penetration enhancers, such as terpenes, anti-oxidants, anti-infectives, anti-inflammatories, growth factors and surfactants.

### EXAMPLES

The invention will now be described by way of example only, with reference to the accompanying drawings, in which:

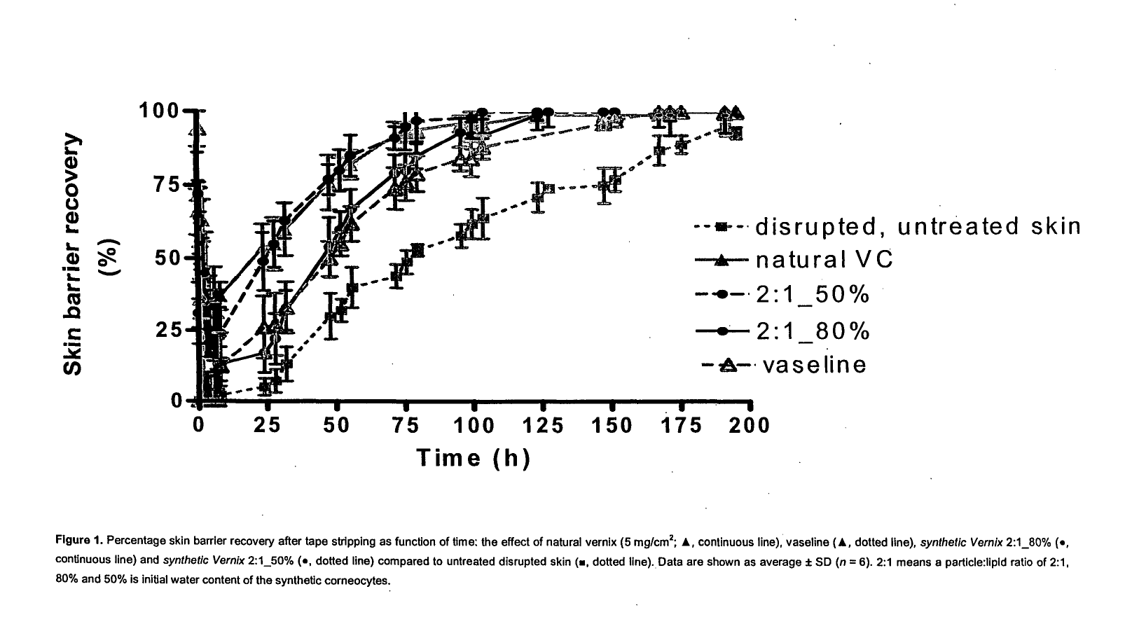

FIG. 1 shows a graph of the percentage skin barrier recovery after tape stripping;

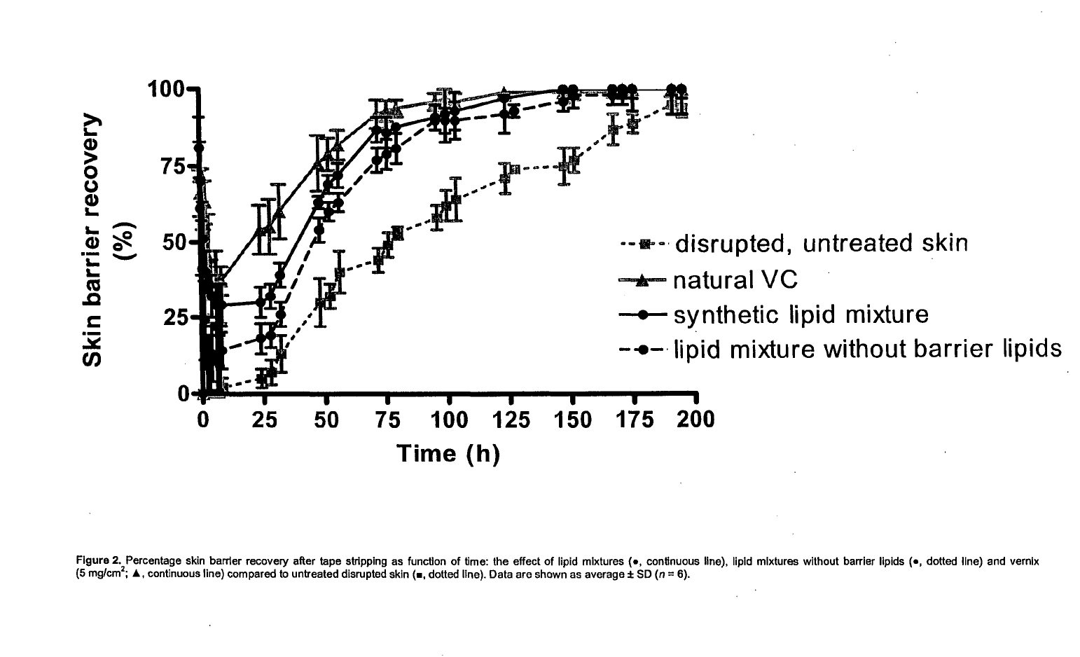

FIG. 2 shows a further graph of the percentage skin barrier recovery after tape stripping;

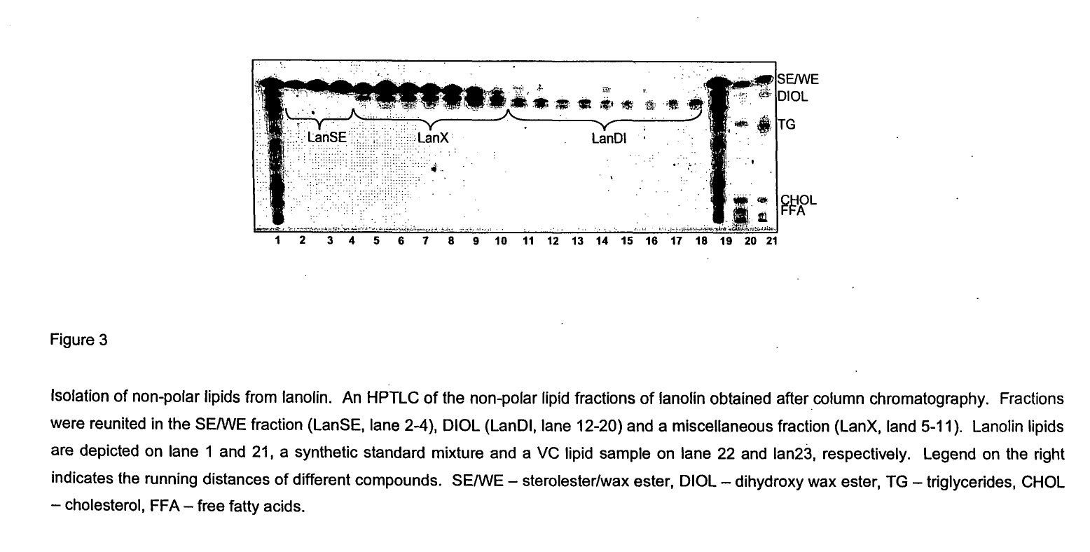

FIG. 3 shows an HPTLC of the non-polar lipid fractions separated from lanolin;

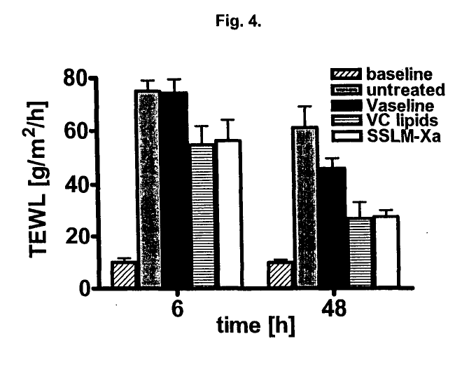

FIG. 4 shows a graph of transepidermal water loss for certain compositions;

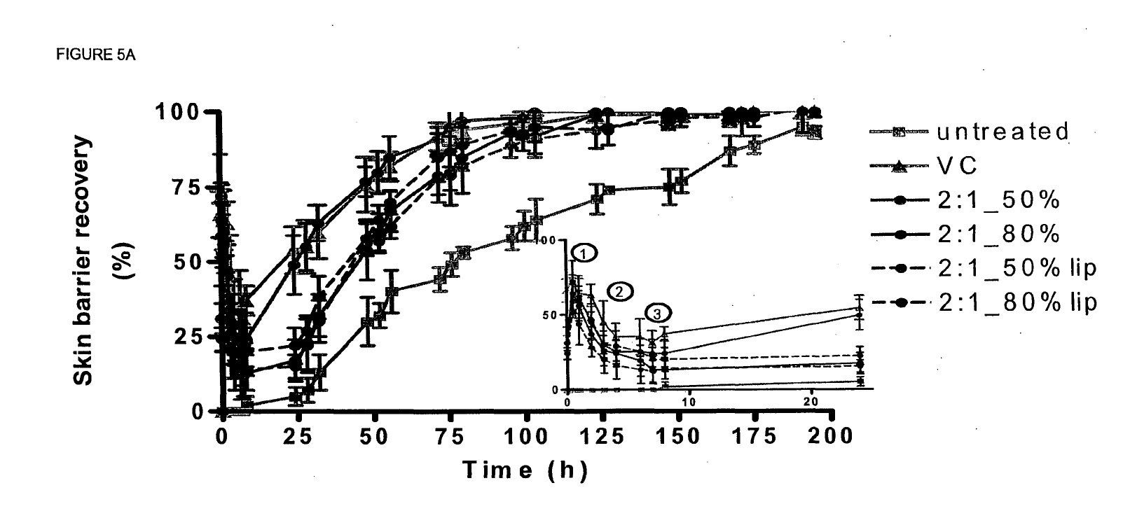

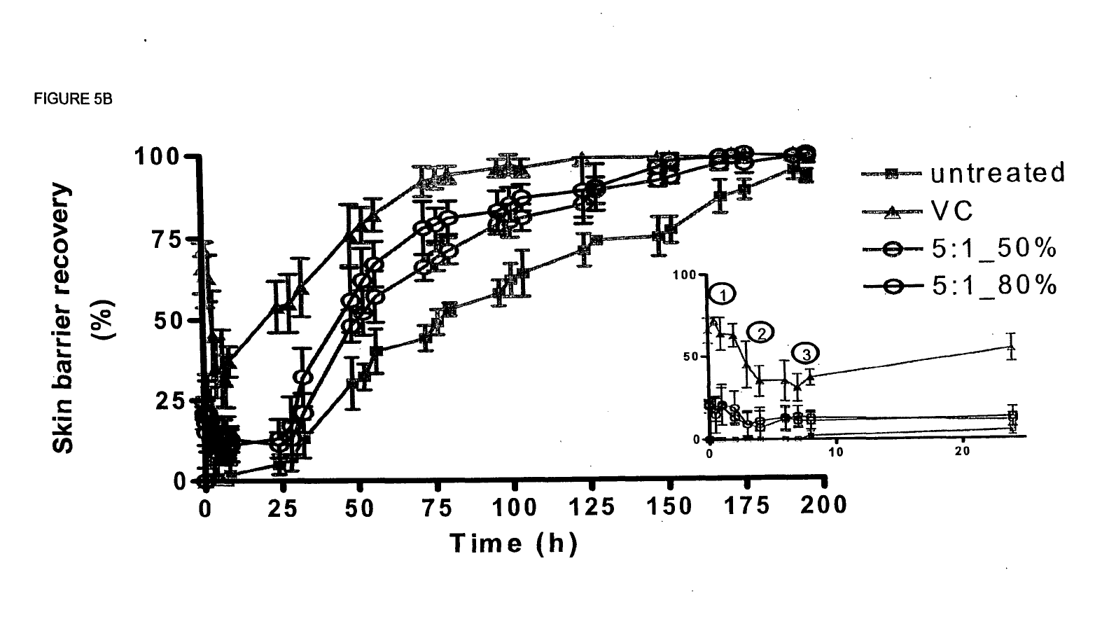

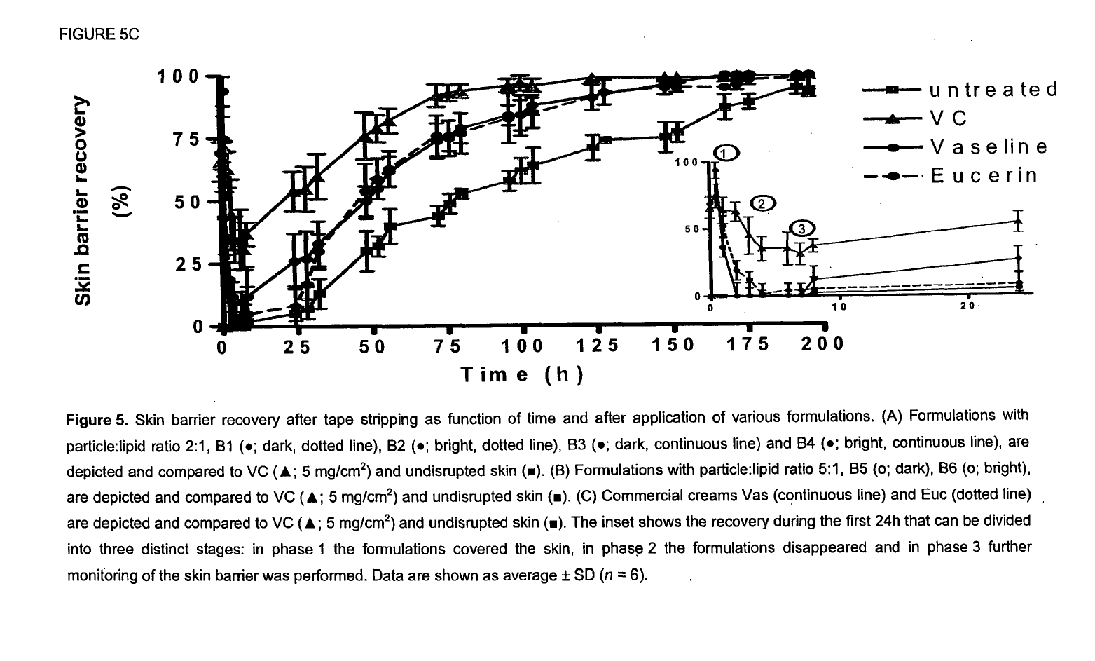

FIG. 5 shows skin barrier recovery of skin after tape stripping and application of certain compositions;

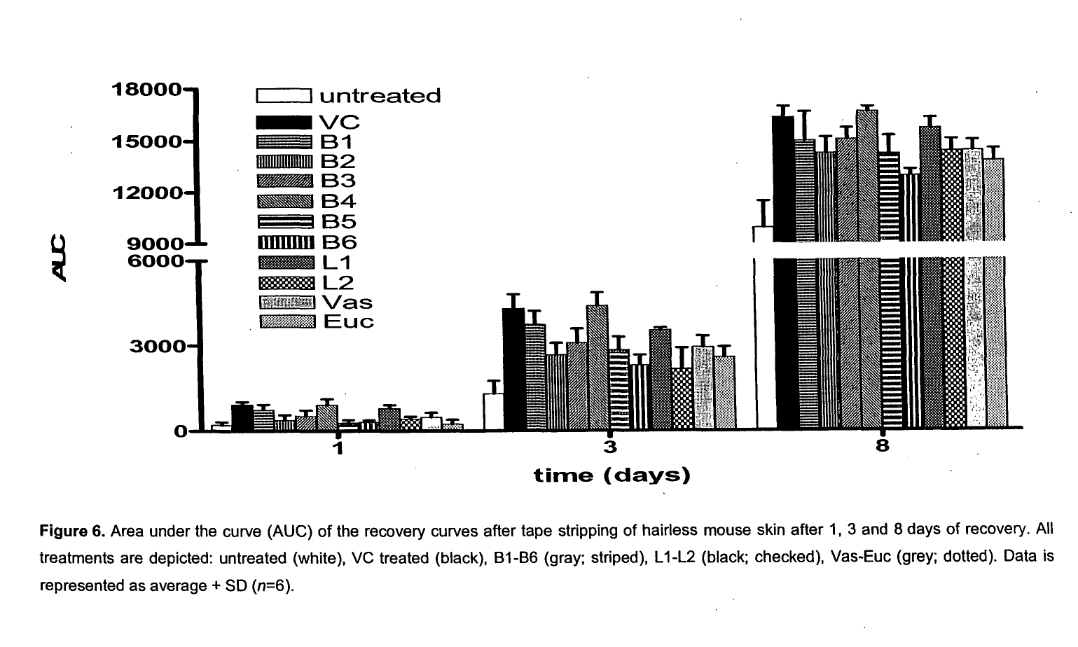

FIG. 6 shows recovery of skin after tape stripping and application of certain compositions; and

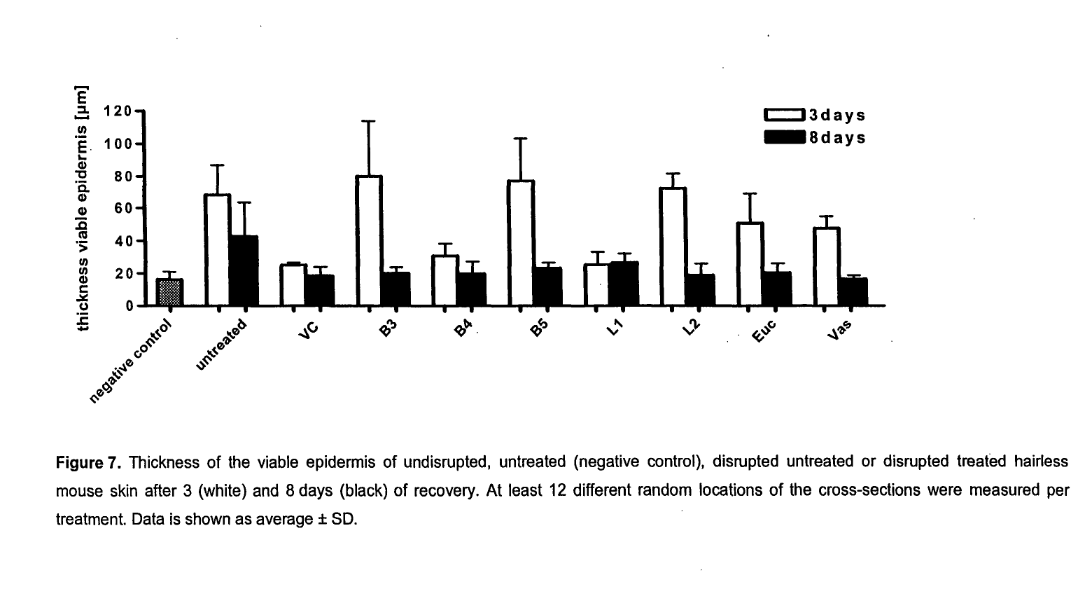

FIG. 7 shows the thickness of the viable epidermis after treatment with certain compositions.

### EXAMPLE 1

The following compositions represent suitable topical compositions for use according to the invention:

TABLE 1

| | | Composition | | | |
|---|---|---|---|---|---|
| | | A | B | C | D |
| | | Synthetic | Synthetic | Synthetic | Synthetic |
| | | Vernix | Vernix | Vernix | Vernix |
| Compound | Description | 2:1_50% | 2:1_80% | 5:1_50% | 5:1_80% |
| Water | | to 100 | to 100 | to 100 | to 100 |
| HyPG-MA* | polymer forming | 6.9 | 11.0 | 22.1 | 15.3 |
| | the particle | | | | |
| Lipids (total) | | (33.3) | (33.3) | (16.7) | (16.7) |
| LanX | combined fractions of | 16.0 | 16.0 | 8.0 | 8.0 |
| | sterol esters, wax | | | | |
| | esters and dihydroxy | | | | |
| | wax esters from | | | | |
| | lanolin (wool wax) | | | | |
| trinervonin | triglycerides | 4.0 | 4.0 | 1.9 | 1.9 |
| triolein | triglycerides | 4.0 | 4.0 | 1.9 | 1.9 |
| tripalmitolein | triglycerides | 4.0 | 4.0 | 1.9 | 1.9 |
| squalene | non-polar alkene | 2.1 | 2.1 | 1.1 | 1.1 |
| cholesterol | sterol | 1.2 | 1.2 | 0.6 | 0.6 |
| ceramide EOS (C30)** | | 0.2 | 0.2 | 0.1 | 0.1 |
| ceramide NS (C24)** | | 0.3 | 0.3 | 0.2 | 0.2 |
| ceramide NP (C24)** | | 0.1 | 0.1 | 0.1 | 0.1 |
| ceramide NP (C16)** | | 0.1 | 0.1 | 0.1 | 0.1 |
| ceramide AS (C24)** | | 0.2 | 0.2 | 0.1 | 0.1 |
| ceramide AP (C24)** | | 0.5 | 0.5 | 0.3 | 0.3 |
| palmitic acid | fatty acid | 0.3 | 0.3 | 0.1 | 0.1 |
| palmitoleic acid | fatty acid | 0.1 | 0.1 | 0.1 | 0.1 |
| stearic acid | fatty acid | 0.1 | 0.1 | 0.1 | 0.1 |
| oleic acid | fatty acid | 0.1 | 0.1 | 0.1 | 0.1 |

*photocross-linkable methacrylated hyperbranched polyglycerol (Mn ~2000 g/mol).
**Ceramide subclasses are denoted by the letter-based system introduced by Motta et al. [S. Motta, M. Monti, S. Sesana, R. Caputo, S. Carelli, R. Ghidoni, Ceramide composition of psoriatic scale, Biochim Biophysic Acta. 1182 (1993) 147-151.) Chemically, a ceramide consists of a sphingoid moiety which can be either sphingosine (S), phytoshphingosine (P) or 6-hydroxysphingosine (H). The fatty acid moiety which is linked to that base is either non-hydroxylated (N), α-hydroxylated (A) or ω-hydroxylated (O). Remarkably, often a C18 fatty acid (high percentage is linoleic acid) is linked to the ω-hydroxy fatty acid moiety of the ceramides which forms an ester (E). The number of carbon atoms (C) of the ceramide acyl-chain is either C16, C24 or C30. In these compositions, the ratio in the name of the composition represents weight ratio of particles to lipid in the composition. The % in the composition name (e.g. 80%, 50%) represents the % by weight of water in the synthetic cell particle. All amounts are in % by weight.

The HyPG-MA particles had a MW of approximately 2000 g/mol. The polymer was modified to provide networks as described in Oudshoorn M H M, Rissmann R, Bouwstra J A, Hennink W E. “Synthesis and characterization of hyperbranched polyglycerol hydrogels.” Biomaterials 2006; 27(32): 5471-5479. Once modified, the polymers are mixed with a certain amount of water (referred to as the initial water content of the particles, e.g. 50% or 80%) and cross-linked with UV using lrgacure 2959 as an initiator, as described in Oudshoorn M H M, Rissmann R, Bouwstra J A, Hennink W E. “Synthesis and characterization of hyperbranched polyglycerol hydrogels.” Biomaterials 2006; 27(32): 5471-5479.

To obtain the small particles with a hexagonal shape and of the desired size, e.g. 30 micron, a photolithography technique was used. That is, a mask and glass plate are separated by spacers with a desired thickness (e.g. 20 microns), and a polymer solution is applied between. UV is applied through the mask; the polymer solution only polymerises on the areas where the mask is open. Un-crosslinked material can then be washed away and the particles are obtained in water. The technique is described in more detail in Oudshoorn M H M, Penterman R, Rissmann R, Bouwstra J A, Broer D J, Hennink W E. “Preparation and characterization of structured hydrogel microparticles based on crosslinked hyperbranched polyglycerol.” Langmuir 2007; 23(23): 11819-11825. Once the particles are obtained in water they are centrifuged, and the supernatant water removed. The cells can then be mixed with the remained of the composition.

### EXAMPLE 2

A common procedure for controlled stratum corneum (SC) damage and repair in an experimental setting is tape stripping [1-4], which removes both intercellular lipids and cellular components of the SC. With this method a variety of skin disturbances and treatments can be studied. Once the barrier function is impaired a homeostatic repair response is initiated within the epidermis, which results in a rapid recovery [1, 5]. By measuring the changes in trans-epidermal water loss (TEWL), the epidermal barrier function can be assessed. It has been shown previously [1, 4, 6, 7] that TEWL measurements offer, via a non-invasive method, a good estimate about the skin barrier status under various conditions. Any alteration in the barrier function will result in modified TEWL measurements.

The purpose of this Example was to determine whether the barrier recovery of disrupted mouse skin would be influenced after the topical application of various products (i.e. Vernix of natural origin, synthetic Vernix etc.). The study showed that our synthetic Vernix increase barrier repair compared to untreated and Vaseline treated skin. Furthermore, variation in composition of the synthetic Vernix affects the barrier recovery rate.

Materials and Methods

Skin Barrier Disruption

Male hairless mice (Skh-1), 7-9 weeks old, were used for the studies. After anaesthesia, the skin of the mice was washed carefully with deionized water prior to marking two areas (˜1 cm², both left and right) on the upper flank of the back of the mice, near the head. An abnormal barrier (severe disruption; defined as TEWL of ≧60 g/m² per h) was induced by tape stripping by a single individual. No scratching of the treated area or any abnormal behaviour was observed during the studies.

Application

Immediately after disruption of the skin barrier, one test area per mice was treated with one single product (listed below in Tables 2 and 3). A single individual applied the samples onto the treatment area with a spatula. Untreated bilateral sites served as controls.

TABLE 2
Applied products on disrupted mouse skin

| | Amount |
|---|---|
| Applied products | (mg/cm²) |
| natural Vernix | 5 |
| natural Vernix | 5 (applied twice) |
| natural Vernix | 15 |
| Vaseline | 5 |
| Synthetic lipid mixture | 5 |
| Synthetic lipid mixture without barrier | 5 |
| lipids | |
| Various synthetic Vernix compositions | 5 |
| including synthetic cells | |

The lipid mixture used in the “Synthetic lipid mixture” is the same as that outlined in Example 1 compositions A and B, i.e. containing 33.3 wt % lipids, and also minus the HyPG-MA particles. The lipid mixture used in the “Synthetic lipid mixture without barrier lipids” is the same as that outlined in Example 1 compositions A and B, i.e. containing 33.3 wt % lipids, minus the HyPG-MA particles and also minus the cholesterol, ceramide and free fatty acid components. The “Various synthetic Vernix compositions including synthetic cells” compositions are as outlined in Example 1 compositions A and B, but including HyPG-MA synthetic cell particles prepared as described in Example 1.

TABLE 3
Composition of the various synthetic Vernix compositions containing
synthetic cells

| | Composition of the various | | |
|---|---|---|---|
| | synthetic Vernix composition | | |
| Name of synthetic | Ratio | Solid content | Particles coated or |
| Vernix composition | particle:lipid | particles | not with lipids |
| 2:1_80% | 2:1 | 20% | Coated |
| 2:1_80% | 2:1 | 20% | Uncoated |
| 2:1_50% | 2:1 | 50% | Coated |
| 2:1_50% | 2:1 | 50% | Uncoated |

The % quoted in the name of the composition is the % water by weight in the particle. The synthetic cells in these compositions were in the form of particles, and were of the same composition as and prepared as outlined in Example 1 above. The synthetic Vernix compositions had a weight ratio of hydrated particle to lipid of 2:1

The coating on the coated particles is a mixture of dioleoyl phosphatidylcholine and dioeoyl trimethylammonium propane, as described in De Geest, Stubbe, Jonas, Van Thienen, Hinrichs, Demeester, De Smedt, “Self-exploding lipid-coated microgels”, Biomacromolecules 2006; 7(1): 373-379,

Evaluation of the Skin

The level of barrier disruption and the repair rate were assessed by measuring TEWL on a daily basis using the TEWA meter TM 210 (Khazaka Courage, Cologne, Germany). The percentage of barrier recovery was calculated using the following formula: 1−((TEWL at indicated time point−average control TEWL)/(TEWL immediately after stripping−average control TEWL))×100%.

Results

The mouse skin was severely disrupted by tape stripping (TEWL of ≧60 g/m² per h). The skin was clearly glistening and an increased redness of the skin was observed indicating skin irritation. Histology confirmed that the stratum corneum was completely removed and that the remaining epidermis was intact. When evaluating the recovery, it was observed that a crust was formed on the disrupted area within a few hours. As a result only 50% barrier recovery was observed within 72 h. Within 8 days the skin barrier was almost completely repaired (˜90%) although some cicatrices developed.

Upon application of natural Vernix on the disrupted skin, it was observed that redness disappeared directly and no crust was formed. The skin recovered much faster and no cicatrices were formed compared to skin which was not treated with Vernix. These observations were confirmed by TEWL measurements; complete recovery was already obtained within 100 h after natural Vernix application, whereas complete recovery only occurred after 200 h at the control site (FIG. 1). Application of an increased dose (i.e. 15 mg/cm²) or multiple applications (i.e. two times 5 mg/cm²; second dose applied 4 h after first dose) of Vernix did not accelerate further the skin barrier recovery. Histology confirmed once more these results (data not shown).

The effect of Vaseline (petrolatum) application on this model was also evaluated. The oil-based ointment Vaseline has been speculated to be occlusive and to increase barrier recovery [8, 9]. It was observed that upon application of Vaseline on disrupted skin the redness did not disappear although crust formation was largely prevented. TEWL measurements showed that after application of Vaseline, recovery was faster than the control area but slower than natural Vernix treated skin (FIG. 1). When applying the various synthetic Vernix compositions, it was observed that the composition 2:1_{—}50% (i.e. synthetic corneocytes comprising of 50% polymer and 50% water) showed similar recovery properties as natural Vernix, whereas the composition 2:1_{—}80% (synthetic corneocytes comprising 20% polymer and 80% water) showed similar results as Vaseline (FIG. 1). In addition, it was observed that the use of lipid-coated particles in the various compositions compared to uncoated particles in this instance did not improve the barrier.

After application of the compositions on disrupted skin, the effect of the lipids alone (without particles) was evaluated. Two different lipid mixtures were evaluated, namely lipid mixtures similar mixture as used in the synthetic Vernix, and lipid mixtures that did not contain the barrier lipids (cholesterol, free fatty acids and ceramides). The results are shown in FIG. 2. It can clearly be observed that lipid mixtures (with or without barrier lipids) increase the barrier recovery although slower than natural Vernix. Moreover it was observed that barrier lipids play an important role; skin barrier recovery of lipid mixtures without barrier lipids is significantly slower compared to lipid mixtures that do contain this class of barrier lipids.

Conclusions

The present study shows a considerable increased skin barrier recovery following topical application of natural Vernix and synthetic Vernix composition 2:1_{—}50% on extensive disrupted skin. Upon application of Vaseline and other types of synthetic Vernix, the skin barrier recovery increased slightly faster compared to the control, but slower than natural Vernix treated skin. Additionally, it was observed that barrier lipids play an important role in the skin barrier recovery.

### REFERENCES

1. Yang L., Mao-Qiang M., Taljebini M., Elias P. M., Feingold K. R. Topical stratum corneum lipids accelerate barrier repair after tape stripping, solvent treatment and some but not all types of detergent treatment. Br J Dermatol 1995, 133(5), 679-685.
2. Uhoda E., Piérard-Franchimont C., Debatisse B., Wang X., Piérard G. Repair kinetics of the stratum corneum under repeated insults. Exog Dermatol 2004, 3, 7-11.
3. Breternitz M., Flach M., Prassler J., Elsner P., Fluhr J. W. Acute barrier disruption by adhesive tapes is influenced by pressure, time and anatomical location: integrity and cohesion assessed by sequential tape stripping. A randomized, controlled study. Br J Dermatol 2007, 156(2), 231-240.
4. van de Kerkhof P. C., de Mare S., Arnold W. P., van Erp P. E. Epidermal regeneration and occlusion. Acta Derm Venereol 1995, 75(1), 6-8.
5. Hachem J. P., Houben E., Crumrine D., Man M. Q., Schurer N., Roelandt T., et al. Serine protease signaling of epidermal permeability barrier homeostasis. J Invest Dermatol 2006, 126(9), 2074-2086.
6. Fluhr J. W., Feingold K. R., Elias P. M. Transepidermal water loss reflects permeability barrier status: validation in human and rodent in vivo and ex vivo models. Exp Dermatol 2006, 15(7), 483-492.
7. Grubauer G., Elias P. M., Feingold K. R. Transepidermal water loss: the signal for recovery of barrier structure and function. J Lipid Res 1989, 30(3), 323-333.
8. Bautista M. I., Wickett R. R., Visscher M. O., Pickens W. L., Hoath S. B. Characterization of vernix caseosa as a natural synthetic Vernix: comparison to standard oil-based ointments. Pediatr Dermatol 2000, 17(4), 253-260.
9. Ghadially R., Halkier-Sorensen L., Elias P. M. Effects of petrolatum on stratum corneum structure and function. J Am Acad Dermatol 1992, 26(3 Pt 2), 387-396.

### EXAMPLE 3

Preparation of Non-Polar Lanolin Extract

The non-polar lipids from (anhydrous) lanolin (ex. Caesar & Lorentz, Bonn, Germany, purchased in hydrous form but subsequently dehydrated) were isolated by means of column chromatography. 32 g Lioprep 60R was dehydrated for 1 hour at 130° C., after which 40 ml chloroform was added. The mixture was then poured into a glass column with a diameter of 20 mm and a length of 420 mm. The pre-column was packed with 4 g dehydrated silica gel. Subsequently, the column was eluted and packed with 100 ml chloroform/hexane 1:1 (v/v). The dry lipid sample was dissolved in the first eluent (see below) and carefully applied on the column. After discarding the death-volume of ˜55 ml, fractions of 3.8 ml were collected during the elution with the following eluents: 150 ml hexane/chloroform/diethyl ether 96:4:0.5 (v/v), 100 ml hexane/chloroform/diethyl ether, 90:6:1 (v/v), 100 ml hexane/chloroform/diethyl ether/dioxan 88:10:2:0.5 (v/v). The lipid fractions were subsequently dried under a gentle stream of nitrogen at 40° C. and analyzed qualitatively by means of HPTLC.

Analysis of the lipid fraction obtained from the column chromatography was performed using one dimensional HPTLC as described in Hoeger et al, “Epidermal barrier lipids in human vernix caseosa; corresponding ceramide pattern in vernix and fetal skin”, Br J. Dematol. 146 (2002) 194-201 and Ponec et al, “New acylceramide in native and reconstructed epidermis”, J. Invest Dermatol. 120 (2003) 581-588. Briefly, 5-50 μg of lipid samples were applied on a rinsed and dehydrated HPTLC plate by means of a Linomat I V (Camag, Muttenz, Switzerland). The non-polar lipids were separated and evaluated by developing the HPTLC plates in hexane/chloroform/diethyl ether/ethyl acetate 48:48:4:1 (VN) for 95 mm. After charring at 170° C., the HPTLC plate was scanned with the Bio Rad GS-710 Calibrated Imaging Densitometer (Hercules, USA) and analysed with Bio Rad software Quantity One.

The results are shown in FIG. 3.

The major non-polar lipid fractions isolated are shown as the sterol ester/was ester fraction (lanes 2 to 4), the dihydroxy wax ester fraction (lanes 12 to 20), and the fraction containing both sterol esters/was esters and dihydroxy wax esters (lanes 5 to 11).

Lanolin lipids are shown in lanes 1 and 21, whilst natural Vernix lipids are shown in lane 23.

### EXAMPLE 4

Various experiments were conducted to determine whether the non-polar lipid fractions of lanolin would mimic the beneficial properties of natural Vernix, and if so which non-polar lipid fraction was most beneficial.

The following test compositions were prepared.

TABLE 4
COMPONENT (wt %,)

| | | | Sterol Ester/ | | | | | | |
|---|---|---|---|---|---|---|---|---|---|
| Compo- | | Sterol Ester/ | Wax Ester and | Dihydroxy | Triglyc- | Modified | Choles- | Free Fatty | |
| sition | Squalene | Wax Ester¹ | dihdroxy wax ester² | Wax Ester³ | eride⁴ | Triyglycerides⁵ | terol⁶ | Acid | Ceramides⁷ |
| 1 | 6.8 | 44.5 | | | 38.1 | | 3.7 | 1.6 | 5.3 |
| 2 | 6.4 | 41.9 | | 6.0 | 35.8 | | 3.5 | 1.5 | 4.9 |
| 3 | 6.4 | | 47.9 | | 35.8 | | 3.5 | 1.5 | 4.9 |
| 4 | 6.8 | 44.5 | | | | 38.1 | 3.7 | 1.6 | 5.3 |
| 5 | 6.4 | 41.9 | | 6.0 | | 35.8 | 3.5 | 1.5 | 4.9 |
| 6 | 6.4 | | 47.9 | | | 35.8 | 3.5 | 1.5 | 4.9 |
| 7 | 5.7 | | 42.6 | | | 31.9 | 7.0 | 3.0 | 9.8 |

¹As per blended fraction from lanes 2 to 4 in Example 3 - LanSE
²As per blended fraction from lanes 5 to 12 in Example 3 - LanX
³As per blended fraction from lanes 12 to 20 in Example 3 - LanDi
⁴A blend of triglycerides 16:0, 16:1, 18:0, 18:1 in the weight ratio 30.4:39.7:5.6:24.3
⁵A blend of triglycerides 16:1, 18:1, 24:1 in the weight ratio 1:1:1
⁶Fatty acids C16:0, C16:1, C18:0 and C19:1 in the weight ratio 53.3:20.0:6.7:20.0.
⁷A blend of ceramides EOS (C30) linoleate, NS(C24), NP(C24), NP(C16), AS(C24), AP(C24) at weight ratios14.6:20.8:8.3:8.3:16.7:31.3%

The components LanSE, LanX and LanDi were examined for their thermotropic behaviour as measured by differential scanning calorimetry (DSC). The measurements were carried out on a Q-1000 calorimeter (TA Instruments, New Castle, Del., USA). Dry lipids (1-5 mg) were transferred into an aluminium pan. Subsequently, the pan was hermetically sealed. After 5 minutes equilibration at 5° C., DSC was performed with a heating rate of 2° C,/min and a modulation of +/−1° C./min up to 50° C.

All components shared some similarities in thermotropic behaviour to VC lipids, but those of LanX (comprising the sterol esters, was esters and dihydroxy wax esters) most closely resembled the behaviour of natural Vernix, in particular in the physiological range.

The thermotropic behaviour of the lipid is important, since a feature of natural Vernix is its strong temperature dependence of its dehydration properties. Natural Vernix has a four times higher dehydration rate at 37° C. compared to room temperature. It is speculated that this contributes the natural Vernix's ability to hydrate a newborn baby's skin in a sustained manner. Hence, it is desirable that a synthetic Vernix composition mimic the thermotropic behaviour of natural Vernix.

Further, the Compositions 1 to 7 in Table 4 were subjected to thermotropic behaviour analysis. Those compositions containing the Triglycerides⁴ batch of triglycerides (containing 64% by weight unsaturated triglycerides) were found to be less close in thermotropic behaviour to natural Vernix compared to those triglycerides labelled Modified Triglycerides⁵ (containing 100% unsaturated triglycerides). This was particularly the case with Composition 6, which contained a mixture of sterol esters and wax esters, and dihydroxy wax esters. The use of more highly unsaturated triglycerides may also be of importance in compositions containing ceramides of synthetic origin which tend to have relatively uniform acyl chain lengths (e.g. C30, C24 and C16).

### EXAMPLE 5

Composition 6 from Example 4 above (SSLM-Xa in FIG. 4) was examined according to a trans-epidermal water loss experiment protocol similar to that outlined in Example 2 above. The results are shown in FIG. 4.

### EXAMPLE 6

In Vivo Study in Mice

Male hairless mice (Skh-1), 7-9 weeks old and 28 g±2 g in weight, were maintained in the animal care facility of the Gorlaeus Laboratories, Leiden University, with temperature- and humidity-controlled room, and fed standard laboratory chow and tap water ad libitum. The animals were anaesthetized using a mixture of Ketamine (150 mg/kg body weight; Nimatek®, Euovet Animal Health B.V., Bladel, The Netherlands) and Xylazine (10 mg/kg body weight; Rompun®, Bayer B. V., Mijdrecht, The Netherlands) by intraperitoneal injection (i.p.). The mice were grouped randomly (six per group), with each group receiving a different treatment.

The skin of the mice was washed carefully with deionized water prior to marking two areas (˜1 cm², both left and right) on the upper flank of the back of the mice, near the head. An impaired skin barrier was induced by sequential tape stripping by a single individual. Tape strips (black D-squame) of ˜1 cm² were cut and applied on the marked areas. The strips were compressed for 5 seconds before being removed in alternated stripping direction. Severe levels of barrier disruption, defined as TEWL of 79±6 g/m² per h (12 tape strips), were induced. After treatment, the mice were housed individually to avoid fight-induced skin injury. No scratching of the treated area or any abnormal behaviour was observed during the studies.

In Vivo Application of Synthetic Vernix Compositions

Immediately after disruption of the skin barrier, one test area per mouse was treated with natural Vernix, Vaseline, Eucerin cum aqua or with one of the synthetic Vernix compositions containing synthetic cells (5 mg/cm²). Additionally, various synthetic Vernix lipid mixtures without synthetic cells were evaluated: synthetic lipid mixtures (synthetic counterpart of isolated natural Vernix lipids) and a similar synthetic mixture without the barrier lipids (ceramides, free fatty acids and cholesterol). All applied formulations are described in Tables 5A and 5B. A single individual applied the samples onto the treatment area with a spatula. The bilateral untreated site served as control.

After application the effect of the various formulations on disrupted skin was evaluated by macroscopic observations, trans-epidermal water loss (TEWL) measurements and histology.

TABLE 5A
Composition of the various synthetic Vernix compositions applied on
disrupted skin

| | | Composition | | |
|---|---|---|---|---|
| | | | Initial water | Lipid |
| | | Particle:lipid | content particles | coated |
| Entry | Sample | ratio | (% w/w) | particles |
| B1¹ | 2:1_80_coated | 2:1 | 80 | Yes |
| B2² | 2:1_50_coated | 2:1 | 50 | Yes |
| B3¹ | 2:1_80 | 2:1 | 80 | No |
| B4² | 2:1_50 | 2:1 | 50 | No |
| B5³ | 5:1_80 | 5:1 | 80 | No |
| B6⁴ | 5:1_50 | 5:1 | 50 | No |

¹Composition B in Example 1, Table 1
²Composition A in Example 1, Table 1
³Composition D in Example 1, Table 1
⁴Composition C in Example 1, Table 1

TABLE 5B
List of the lipid mixtures, natural Vernix and commercially available
creams applied on the disrupted skin.

| Entry | Sample |
|---|---|
| L1 | Synthetic lipid mixture |
| L2 | Synthetic mixture without |
| | barrier lipids |
| VC | Natural Vernix caseosa |
| Vas | Vaseline |
| Euc | Eucerin |

⁵ Composition 6 in Example 4, Table 4
⁶ Lipid mixture comprising 7.1% squalene, 53.2% sterol ester/wax ester and 39.7% modified triglycerides

Macroscopic Observations

The various formulations had different effects on redness and crust formation in time. Therefore, the extent of redness and crust formation was classified into four different levels (i.e. obviously (++), intermediately (+), slightly (+/−) or absent (−)). The degree of redness and crust formation was independently scored by 3 different investigators (Table 6).

The disrupted untreated site was clearly glistening and red following tape stripping, after which a crust was formed. Upon application of natural Vernix, redness disappeared in a few minutes and crust formation was prevented. Application of the various compositions on the disrupted skin resulted in different observations: slight to intermediate irritation (redness) and crust formation was observed for all synthetic compositions (Table 6). The compositions B2, B3, B5 and B6 showed predominantly intermediate crust formation, whereas B1 showed only slight redness and crust formation.

However, it was clearly observed that application of B4 prevented largely both redness and crust formation. In addition it was observed that L1 showed similar results as B4: redness and crust formation was largely prevented. When omitting the barrier lipids (i.e. cholesterol, fatty acids, and ceramides) from the lipid matrix, obtaining the lipid mixture L2, redness and crust formation were clearly visible, indicating the important effect of barrier lipids. For comparison also the commercially available creams Vas and Euc were evaluated. Both creams prevented only partially redness and crust formation.

TABLE 6
Rating of redness and crust formation of the disrupted sites followed in
time. The average evaluation of digital pictures scored by three
independent investigators is presented.

Rating in time*

| Treatment | 8 h | 1 d | 3 d | 5 d |
|---|---|---|---|---|
| Untreated | ++ | ++ | ++ | + |
| VC | − | − | − | − |
| B1 | +/− | +/− | +/− | +/− |
| B2 | +/− | +/− | + | − |
| B3 | +/− | +/− | + | − |
| B4 | − | +/− | +/− | − |
| B5 | +/− | +/− | + | − |
| B6 | +/− | + | + | − |
| Vas | +/− | +/− | +/− | − |
| Euc | +/− | +/− | +/− | − |
| L1 | − | +/− | +/− | − |
| L2 | +/− | +/− | +/− | − |

*Redness and crust formation on skin were assessed as obviously (++), intermediately (+), slightly (+/−) or absent (−)

Conclusion

Composition B4 had the best results prevented largely both redness and crust formation. In addition, the synthetic lipid mixture (L1) showed similar results as B4 whereas the commercial creams were largely inferior to these formulations (i.e. B4 and L1).

Trans-Epidermal Water Loss (TEWL) Measurements

Initially the effect of the composition mimicking most closely the properties (in terms of components and water content in the hydrogels) of natural Vernix, although with a lower particle/lipid ratio (i.e. 2:1), on barrier recovery was studied. This composition, referred to as composition 61, had a particle/lipid ratio of 2:1 using lipid coated particles with an initial water content of 80% (w/w). It was observed that upon application of this composition B1 (5 mg/cm²) on disrupted skin, complete recovery occurred already within ˜100 h instead of ˜200 h for untreated disrupted skin (FIG. 6A). The inset (FIG. 5A) shows the initial recovery period (phase 1) where a rapid barrier recovery was observed (TEWL decreased from 79±6 g/m²/h to 38±6 g/m²/h). Visually, the composition B1 disappeared within 3 to 4 h after which the TEWL increased to 56±5 g/m²/h (barrier recovery of 34±6%; phase 2, FIG. 5A).

Further monitoring of the skin barrier (phase 3, FIG. 5A) showed complete recovery within 100 h. Upon application of composition B1, the initial recovery was similar to VC treated skin, whereas the recovery period between 10 and 75 h was slower. Complete recovery however, occurred within the same time span (i.e. 100 h) as natural Vernix treated skin. When using synthetic corneocytes with an initial water content of 50% (w/w) and maintaining other components equal, a similar barrier recovery profile as composition B1 was obtained (FIG. 5A). A comparable skin barrier repair outcome was also obtained (FIG. 5A) when applying composition B3, where uncoated particles were used maintaining the particle/lipid ratio of 2:1 and the initial water content of 80% (w/w; table 1), on the disrupted skin. A composition composed of uncoated synthetic corneocytes with 50% (w/w) initial water content and particle/lipid ratio of 2:1 (composition B4), showed a slightly different profile compared to compositions B1-B3; the complete barrier recovery profile was similar to natural Vernix treated skin (FIG. 5A).

It was observed that compositions with a 5:1 particle/lipid ratio showed a more dense and random particle distribution similar to natural Vernix. The topical application of compositions with a particle/lipid ratio of 5:1 with an initial water content of 80% (w/w) or 50% (w/w) (compositions B5 and B6, respectively) was also evaluated. Lipid coating on the particles was omitted as this did not increase barrier recovery for the other formulations (FIG. 5A). TEWL measurements showed a complete recovery within 150 h after application of both compositions (FIG. 5B). Application of B5 and B6 (5 mg/cm²) decreased only slightly the TEWL from 79±6 g/m²/h to 62±7 and 61±8 g/m²/h, respectively (barrier recovery increased to 20±7% and 21±8%, respectively, indicated as phase 1 in FIG. 5B. Within 1 to 2 h the compositions B5 and B6 disappeared visually, the damaged skin was visible again and the TEWL increased to 69±4 and 71±6 g/m²/h, respectively (phase 2, FIG. 5B).

Further skin barrier repair was monitored (phase 3, FIG. 5B) until complete recovery was achieved (i.e. 150 h). Apparently, increasing the water content of the compositions did not influence skin barrier recovery: neither an increase in initial water content of the particles (i.e. B5 compared to B6; FIG. 6B) nor an increase in the amount of particles (e.g. B5 compared to B3; FIG. 6) improved further the skin barrier repair. TEWL measurements showed that application of the lipid mixture (without the synthetic corneocyte cells) L1 on the disrupted skin resulted in a similar barrier recovery as observed for the compositions B1-B3. Application of L2 on disrupted skin decreased the barrier recovery: complete barrier recovery was obtained within 150 h compared to 100 h for L1.

Application of Vas and Euc (5 mg/cm²) immediately restored the barrier function of the skin (FIG. 5C) demonstrating the occlusive properties. Vas and Euc disappeared visually within 2 h and 4 h, respectively. As a result the TEWL increased again to 77±7 g/m²/h; for untreated disrupted skin the TEWL was in the same range. Subsequent monitoring of the skin barrier showed complete recovery occurred within 150 h. Since the disrupted, untreated skin showed nearly complete recovery within 200 h, application of either Vas and or Euc did enhance slightly barrier recovery. However, when comparing the effect of Vas and Euc to natural Vernix, barrier recovery was slower (i.e. 150 h compared to 100 h, respectively).

In order to evaluate the recovery curves of the various treatments in a statistic manner, the areas under the curves (AUCs) of the individual treatments were calculated and compared. The results after 1, 3 and 8 days of recovery are presented in FIG. 6. In general, the same trends can be observed for all 3 time points: disrupted untreated skin (white bars) exhibits the lowest AUC compared to disrupted treated (applied for all treatments) skin, indicating the lowest recovery rate. Moreover, after 8 days natural Vernix showed significant better recovery than Vas and Euc but no significant difference to the best composition, i.e. B4. In turn, B4 demonstrated an improved recovery versus B2, B5, B6, L2, Vas and Euc. However, no significant difference between B4 and L1 was observed after 8 days although the difference was significant in the initial phase (3 days).

Conclusion

These results indicate that for an improved barrier recovery several aspects are important for formulations: I) The presence of barrier lipids (L1 vs. L2) promotes barrier repair, as was demonstrated previously by several other investigators [1, 2]. II) Occlusion of impaired skin (i.e. application of Vas) only enhances recovery to a small extent. III) Initial high water release from the compositions results in more enhanced barrier repair compared to the sustained water release from the pre-coated microgels (B4 vs. B2). IV) The balanced ratio of particles, water and the lipids are of crucial importance.

Histology

Morphological features of composition-treated skin after 3 and 8 days were examined. Compositions B3, B4 and B5 were chosen as representative for this purpose. After 3 days, the presence of 3-5 corneocyte layers was observed for all compositions. However, the viable epidermis was largely thickened for B3 and B5, similar to disrupted, untreated skin, which is an indication for hyperproliferation. B4 showed a normal thickness of the viable epidermis indicating a further stage in the healing process. After 8 days, the various treatments showed similar results in stratum corneum (SC) and viable epidermis appearance compared to normal hairless mouse skin. In addition, the average thickness of the viable epidermis was determined by measuring 12 random locations of the biopsies.

The data obtained 3 and 8 days after treatment are presented in FIG. 7. After 3 days, the viable epidermis of B3 and B5 was up to 4 times thicker compared to the negative control. Upon treatment with B4, however, the thickness of the epidermis after 3 days was 31.0±8.9 μm and comparable to natural Vernix treated skin (25.2±4.8 μm). After 8 days, the thickness of the epidermis of all treated areas was similar to undisrupted untreated skin. In comparison, the disrupted untreated skin still showed a 2.1 times thickened epidermis.

The histological assessment also indicates an improved recovery with L1 but not with L2. Upon application of L2, the viable epidermis was thicker compared to L1-treatment (not shown). In addition, a very thick SC (10 layers) is already observed after 3 days of recovery for L1 whereas L2 only showed 3 layers. After 8 days, the SC and thickness of epidermis with both treatments are similar to normal skin (data not shown).

Vas and Euc treated skin already showed complete SC (5-6 layers) 3 days after recovery (not shown). After 8 days no major differences in SC could be observed on Vas and Euc treated skin. However, it was observed that both Vas and Euc showed thickened viable epidermis after 3 days of recovery (FIG. 7), which is an indication for hyperproliferation. After 8 days, the thickness of the epidermis of the Vas and Euc treated areas was similar to undisrupted untreated skin. In comparison, the disrupted untreated skin still showed a 2 times thickened epidermis.

Conclusion

These results indicate that after disruption of the skin epidermal thickening is observed. A normal epidermis was already observed 3 days after treatment with the formulations B4 and L1 and natural Vernix whereas all other formulations only showed an improvement 8 days after treatment. This indicates the benefit of these formulations (i.e. B4, L1 and natural Vernix) for a faster barrier recovery.

1. A topical composition comprising the isolated non-polar lipid fraction of lanolin, and one or more additional compounds selected from ceramides, cholesterol and free fatty acids.

2. A topical composition according to claim 1 wherein the additional compounds present in the composition comprise any pair of compounds selected from ceramides, cholesterol and free fatty acids.

3. A topical composition according to claim 1, wherein the additional compounds present in the composition comprise ceramides, cholesterol and free fatty acids.

4. A topical composition according to claim 1, wherein the topical compositions comprise at least 30% by weight lipids.

5. A topical composition according to claim 1, wherein the non-polar lipid fraction is sterol esters, wax esters and dihydroxy wax esters.

6. A topical composition according to claim 1, wherein the ceramide is a synthetic ceramide.

7. A topical composition according to claim 1, wherein the topical composition additionally contains synthetic cells.

8. A topical composition according to any claim 7, wherein the synthetic cells comprise a hydrogel.

9. A topical composition according to claim 7, wherein the synthetic cells comprise a hyperbranched polyglycerol, preferably having a molecular weight of 1,000 to 30,000.

10. A topical composition according to claim 1 wherein the synthetic cell incorporates a benefit material.

11. A topical composition according to claim 7, wherein the synthetic cell has length, width or depth in the range 1 to 100 microns.

12. A topical composition according claim 7, wherein the ratio of hydrated synthetic cell to lipid is in range 8:1 to 1:1.

13. A topical composition according to claim 1, additionally comprising a 0.1% to 35% by weight protein.

14. A topical composition according to claim 1, wherein the composition additionally comprises one or both of added squalene and added triglyceride.

15. A topical composition according to claim 4, wherein the added triglyceride comprises at least 65% by weight unsaturated triglyceride.

16. A topical composition comprising the isolated non-polar lipid fraction of lanolin, and one or more additional compounds selected from cermides, cholesterol and free fatty acids for use as an active therapeutic substance.

17. A topical composition comprising the isolated non-polar lipid fraction of lanolin, and one or more additional compounds selected from ceramides, cholesterol and free fatty acids for use
to improve barrier formation and repair and to act as protective layer (for superficial wounds where stratum corneum is largely deficient or even absent)
to exhibit anti-infective, anti-oxidant or skin hydrating benefits (for skin where stratum corneum is still present; such as dry skin).
